(19) United States
(12) Patent Application Publication
(10) Pub. No.: US 2017/0061096 A1
(43) Pub. Date: Mar. 2, 2017

(54) WORKFLOW DRIVEN BARCODE GENERATION FOR INFUSION PUMPS

(71) Applicant: CERNER INNOVATION, INC., KANSAS CITY, KS (US)

(72) Inventors: LISA KELLY, OVERLAND PARK, KS (US); JUDITH A. ZAKUTNY, OLATHE, KS (US); BRYAN MUEHLMEIER, OVERLAND PARK, KS (US)

(21) Appl. No.: 14/841,086

(22) Filed: Aug. 31, 2015

(51) Int. Cl. G06F 19/00; A61B 90/90; G06K 19/06; A61B 5/117

(52) U.S. Cl. CPC G06F 19/3468; A61B 5/117; A61B 90/90; G06F 19/327; G06K 19/06028

(57) ABSTRACT

Methods, computer systems and computer readable media for generating workflow driven barcodes for medical devices are provided. In embodiments, after an identification of a patient and an infusion has been received and communication has been established between an infusion device and an electronic medical record associated with the patient, a digital barcode is provided via a user interface of an infusion device. Once the barcode is scanned, the order is communicated to the infusion device. After a confirmation is received from the clinician, the infusion begins.

### BACKGROUND

Infusion pumps infuse fluids, medications and/or nutrients into the circulatory system of an individual or patient. The infusions may be intravenous, arterial, epidural and the like. Infusion pumps can administer injections continuously, intermittently, or upon patient request. Infusion pumps are used by clinicians for patients when more accuracy is needed than with manually adjusted gravitational administration of fluids into a patient's circulatory system. Infusions pumps can be used for infusion of a variety of fluids and medications including, but not limited to anesthesia, chemotherapy, IV drugs, blood transfusions and the like.

In a typical workflow, a clinician must follow a particular order before certain medical devices that are used to treat or care for a patient, such as infusion pumps, can begin treating a patient. When the particular order is not followed, the devices enter an error state that causes the clinician to have to reset the device and start the process over. However, in many instances, even when the clinician follows the appropriate order, communication between the pump and an infusion application (or healthcare information system) may not yet be established. If, for example, the clinician scans the infusion pump prior to communication being established, the clinician receives an error in the scanning workflow. In this scenario, the clinician has to make a decision to manually program the infusion pump and manually document or understand the error and wait for the pump to come on-line. This delay can affect timeliness and effectiveness of patient care and may also frustrate the clinician, the patient, and family members of the patient.

### SUMMARY

Embodiments of the present invention generally relate to workflow driven barcode generation for infusion pumps. After an identification of a patient associated with an order for an infusion and an identification of the infusion being administered to the patient by an infusion device is received, a digital barcode is provided via a user interface of the infusion device. The digital barcode is not provided via the user interface until the patient and the infusion are identified and communication has been established between the infusion device and an electronic medical record associated with the patient. Once the barcode is scanned, the order is communicated to the infusion device. After a confirmation is received from the clinician, the infusion begins.

Accordingly, in one aspect, an embodiment of the present invention is directed to one or more computer storage media storing computer-useable instructions that, when used by one or more computing devices, cause the one or more computing devices to perform operations. The operations include receiving an identification of a patient, the patient associated with an order for an infusion. The operations also include receiving an identification of the infusion being administered to the patient by an infusion device. The operations further include providing, via a user interface of the infusion device, a graphical representation of a barcode, wherein the barcode is not provided via the user interface until the patient and the infusion are identified and communication has been established between the infusion device and an electronic medical record associated with the patient. The operations also include, upon the barcode being scanned, communicating the order to the infusion device. The operations further include receiving a confirmation from a clinician to begin the infusion.

In another embodiment, an aspect is directed to a computer-implemented method in a clinical computing environment. The method includes establishing, via a first computing process, communication between an infusion device and an electronic medical record associated with a patient. The method also includes providing, via a second computing process, a graphical representation of a barcode on a display of the infusion device, wherein the barcode is not provided via the user interface until the patient and the infusion are identified and communication has been established. The method further includes upon the barcode being scanned, communicating, via a third computing process, the order to the infusion device. The method also includes receiving, from a fourth computing process, a confirmation from a clinician to begin the infusion. Each computing process is performed by one or more computing devices.

A further embodiment is directed to a system comprising: one or more processors; and one or more computer storage media storing instructions that, when used by the one or more processors, cause the one or more processors to: receive an identification of a patient, the patient associated with an order for an infusion; receive an identification of the infusion being administered to the patient by an infusion device; establish communication with the infusion device and an electronic medical record associated with the patient; provide a graphical representation of a barcode on a display of the infusion device, wherein the barcode is not provided via a user interface until the patient and the infusion are identified and communication has been established; upon the barcode being scanned, communicate the order to the infusion device; and receive a confirmation from a clinician to begin the infusion.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

Embodiments are described in detail below with reference to the attached drawing figures, wherein:

FIG. 1 is a block diagram of an exemplary computing environment suitable to implement embodiments of the present invention;

FIG. 2 is an exemplary system architecture suitable to implement embodiments of the present invention;

FIG. 3 is a flow diagram showing a method for providing workflow driven digital barcodes in accordance with embodiments of the present invention; and

FIG. 4 is a flow diagram showing a method for providing workflow driven digital barcodes in accordance with embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

As mentioned above, a clinician typically must follow a very particular order in a workflow before certain medical devices that are used to treat or care for a patient, such as infusion pumps, can begin treating a patient. If the particular order is not followed, an error is received that requires the clinician to have to reset the device and start the process over. For example, in the case of an infusion pump, a typical workflow includes: 1) scan the patient's wristband; 2) scan the medication; 3) scan the infusion pump; 4) review and confirm infusion characteristics on the infusion pump; 5) sign the order. Even when the clinician follows the appropriate order, communication between the pump and an infusion application (or healthcare information system) may not yet be established. If the clinician scans the infusion pump prior to communication being established, the clinician receives an error. In this scenario, the clinician has to reset the infusion pump and start over at the beginning of the workflow. This delay can affect timeliness and effectiveness of patient care and may also frustrate the clinician, the patient, and family members of the patient.

Embodiments of the present invention generally relate to workflow driven barcode generation for infusion pumps. An identification of a patient associated with an order for an infusion and an identification of the infusion being administered to the patient by an infusion device is received. A digital barcode is provided via a user interface of the infusion device after the identification of the patient and the infusion are received and communication has been established between the infusion device and an electronic medical record associated with the patient. Once the barcode is scanned, the order is communicated to the infusion device. After a confirmation is received from the clinician, the infusion begins.

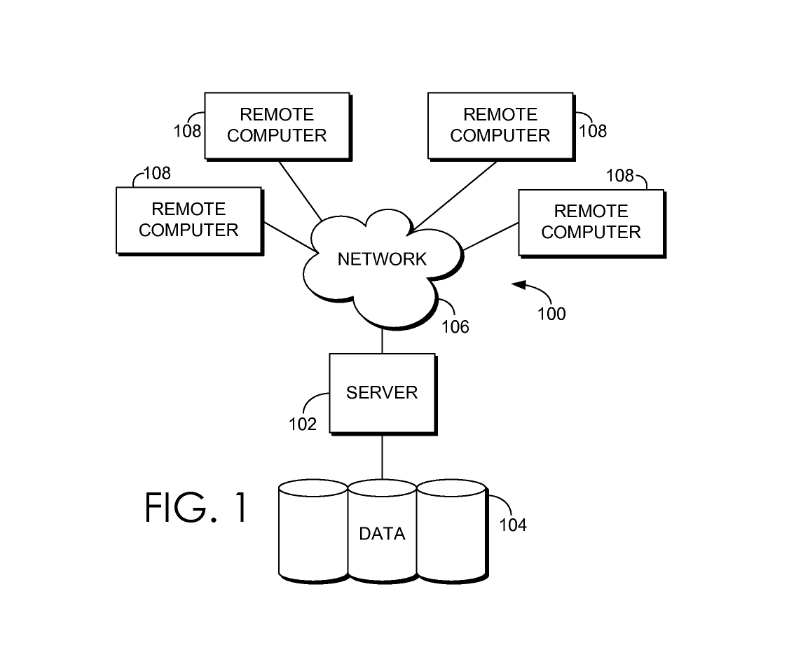

Referring to the drawings in general, and initially to FIG. 1 in particular, an exemplary computing system environment, for instance, a medical information computing system, on which embodiments of the present invention may be implemented is illustrated and designated generally as reference numeral 100. It will be understood and appreciated by those of ordinary skill in the art that the illustrated medical information computing system environment 100 is merely an example of one suitable computing environment and is not intended to suggest any limitation as to the scope of use or functionality of the invention. Neither should the medical information computing system environment 100 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer storage media including, by way of example only, memory storage devices.

With continued reference to FIG. 1, the exemplary medical information computing system environment 100 includes a general purpose computing device in the form of a server 102. Components of the server 102 may include, without limitation, a processing unit, internal system memory, and a suitable system bus for coupling various system components, including database cluster 104, with the server 102. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server 102 typically includes, or has access to, a variety of computer readable media, for instance, database cluster 104. Computer readable media can be any available media that may be accessed by server 102, and includes volatile and nonvolatile media, as well as removable and non-removable media. By way of example, and not limitation, computer readable media may include computer storage media and communication media. Computer storage media may include, without limitation, volatile and nonvolatile media, as well as removable and nonremovable media implemented in any method or technology for storage of information, such as computer readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 102. Computer storage media does not comprise signals per se. Communication media typically embodies computer readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer readable media.

The computer storage media discussed above and illustrated in FIG. 1, including database cluster 104, provide storage of computer readable instructions, data structures, program modules, and other data for the server 102.

The server 102 may operate in a computer network 106 using logical connections to one or more remote computers 108. Remote computers 108 may be located at a variety of locations in a medical or research environment, for example, but not limited to, clinical laboratories, hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as surgeons, radiologists, cardiologists, and oncologists, emergency medical technicians, physicians' assistants, nurse practitioners, nurses, nurses' aides, pharmacists, dieticians, microbiologists, laboratory experts, genetic counselors, researchers, students, office assistants and the like. The remote computers 108 may also be physically located in non-traditional medical care environments so that the entire health care community may be capable of integration on the network. The remote computers 108 may be personal computers, servers, routers, network PCs, peer devices, other common network nodes, or the like, and may include some or all of the components described above in relation to the server 102. The devices can be personal digital assistants or other like devices.

Exemplary computer networks 106 may include, without limitation, local area networks (LANs) and/or wide area networks (WANs). Such networking environments are commonplace in offices, enterprise-wide computer networks, intranets, and the Internet. When utilized in a WAN networking environment, the server 102 may include a modem or other means for establishing communications over the WAN, such as the Internet. In a networked environment, program modules or portions thereof may be stored in the server 102, in the database cluster 104, or on any of the remote computers 108. For example, and not by way of limitation, various application programs may reside on the memory associated with any one or more of the remote computers 108. It will be appreciated by those of ordinary skill in the art that the network connections shown are exemplary and other means of establishing a communications link between the computers (e.g., server 102 and remote computers 108) may be utilized.

In operation, a user may enter commands and information into the server 102 or convey the commands and information to the server 102 via one or more of the remote computers 108 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the server 102. In addition to a monitor, the server 102 and/or remote computers 108 may include other peripheral output devices, such as speakers and a printer.

Although many other internal components of the server 102 and the remote computers 108 are not shown, those of ordinary skill in the art will appreciate that such components and their interconnection are well known. Accordingly, additional details concerning the internal construction of the server 102 and the remote computers 108 are not further disclosed herein.

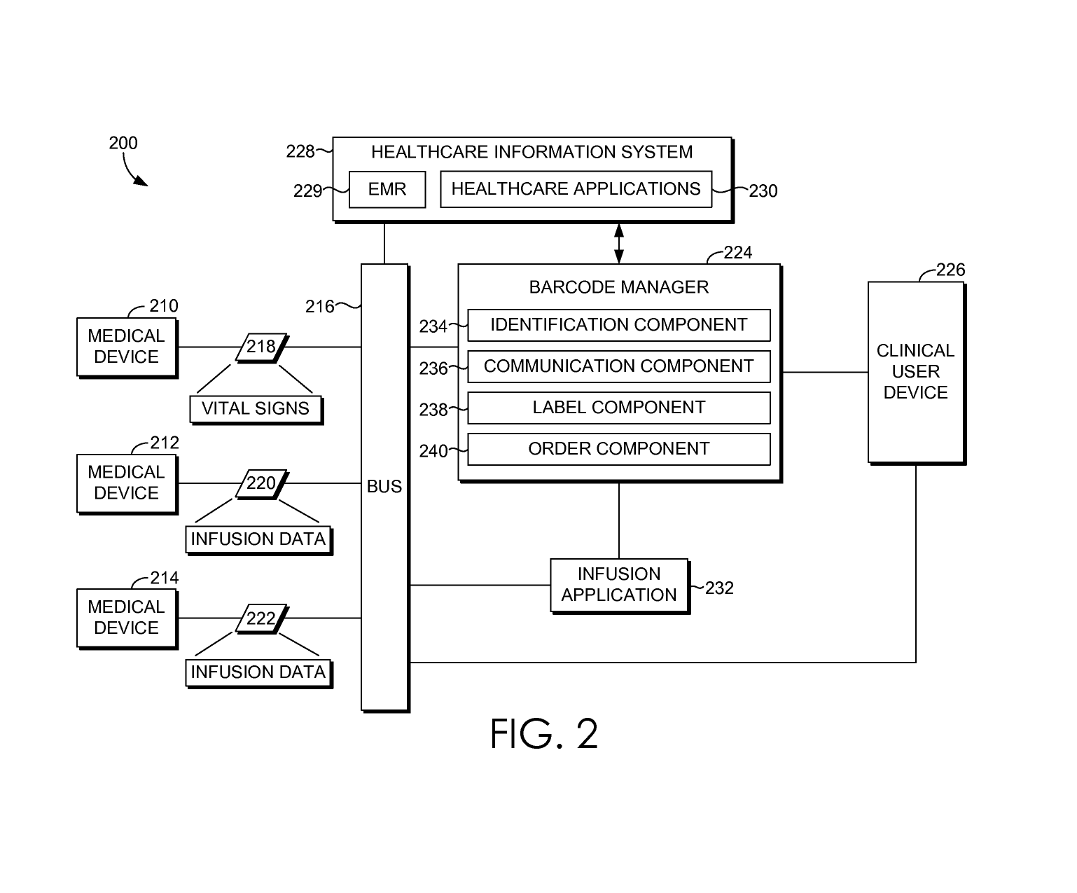

Turning now to FIG. 2, a schematic diagram depicts an operating environment, identified generally by reference numeral 200, suitable to practice an embodiment of the present invention. FIG. 2 includes various components that communicate with one another, including medical device 210, 212, 214, clinical user devices 226, bus 216, barcode manager 224, healthcare information system 228 and infusion application 232. In one embodiment of the present invention, clinical user device is a barcode scanner. The barcode scanner may be utilized to scan a patient's wristband, scan a medication, and/or scan a digital barcode provided by medical devices 210, 212, 214. Data may be received from barcode scanner by barcode manager. Before describing in more detail how these components communicate, each component will be generally described.

In an embodiment of the present invention, medical device 210 might include cardiac monitors, ventilators, balloon pumps, patient beds, sequential-compression devices, electronic security devices, and vital-sign detecting devices. Medical device 210 may generate various data (e.g., measured heart rate) that, as described in more detail below, is communicated to other components (e.g., bus 216) of operating environment 200. Moreover, medical device 210 might also receive information from components of operating environment 200.

In another embodiment of the present invention, medical devices 212, 214 might include infusion pumps. Infusion pumps may infuse fluids, medications and/or nutrients into the circulatory system of an individual or patient. The infusions may be, but are not limited to, intravenous, arterial, epidural and the like. Infusion pumps can administer injections continuously, intermittently, or upon patient request. Infusion pumps are used by clinicians for patients when more accuracy is needed than with manually adjusted gravitational administration of fluids into a patient's circulatory system. Infusions pumps can be used for infusion of a variety of fluids and medications including, but not limited to anesthesia, chemotherapy, IV drugs, blood transfusions and the like. The fluid, medication and/or nutrients are typically contained in an infusion container, such as an infusion bag. It will be appreciate that any type container may be utilized to hold the infusion fluid, medication and/or nutrients. Infusion pumps 212 and 214 generate various data, including, but not limited to, remaining volume of infusion (e.g., amount remaining in fluid container), rate of infusion (e.g., how fast fluid is being infused), alerts (e.g., air in line, maintenance of pump needed, high backpressure, low infusion, occlusion, or pump stopped). This data is communicated to other components (e.g., bus 216) of operating environment 200. Moreover, infusion pumps 212 and 214 might also receive information from components of operating environment 200.

Healthcare information system 228 includes an integrated system of healthcare-related information that is usable by a healthcare facility to operate and provide patient care. For example, healthcare information system 228 includes an electronic medical record 229 (also referred to herein as “EMR”) and a healthcare applications component 230. EMR 229 includes an electronic version of patient records including information for the patient, such as medication and infusion orders, tasks, images, examination reports, testing and lab results, medical history, etc. Healthcare applications component 230 includes information that is input and provided at a patient's point-of-care (e.g., patient bedside) to assist healthcare professionals to provide appropriate care. An exemplary applications component 230 includes a patient order entry component for entering electronic healthcare orders for a patient. In an embodiment of the present invention, healthcare information system 228 receives information from other components, as will be described in more detail below. Moreover, healthcare information system 228 might also provide information that is communicated to other components of operating environment 200.

Clinical user devices 226 include devices that are used within a healthcare facility to receive, display and send information. Exemplary clinical user devices 226 include any device capable of scanning a barcode. In this way, clinical user devices 226 may be used by a clinician to scan a patient's wristband, scan a medication, and/or scan a medical device. In some embodiments, clinical user devices 226 receive information from other components of operating environment 200 based on location or proximity. In this way, clinical user devices 226 may receive information associated with a patient's wristband, a medication, or a medical device by being in the same location or in close proximity (e.g., Bluetooth or RFID range) and may not require physically scanning a barcode. Moreover, clinical user devices 226 might also receive inputs from a clinician that are communicated to other components of operating environment 200. Clinical user devices 226 also communicate to other components of operating environment 200 requests to receive additional information. For example, clinical user device 226 might communicate information to barcode manager 224, infusion application 232, HIS 228, EMR 229, healthcare application 230, and medical devices 210, 212 and 214.

Infusion application 232 is an electronic application for receiving medication orders, such as infusion orders, to be filled. An exemplary pharmacy system is Cerner Millennium Pharmnet by Cerner Corporation, Kansas City Missouri. Typically orders for medications, fluids and nutrients to be filled by a pharmacist are displayed in the pharmacy or pharmacy IV room. The pharmacist can use this information to drive the pharmacy workflow and make sure the necessary medication orders are filled. In another embodiment, infusion application 232 may be an automated pharmacy dispensing system such as Cerner RXStation by Cerner Corporation of Kansas City, MO. The automated pharmacy system may be an apparatus pre-loaded with medication, fluids and/or nutrients that may be dispensed to fill patient orders.

As previously indicated, and as depicted in FIG. 2, each of medical devices 210, 212, 214, healthcare information system 228, barcode manager 224, clinical user devices 226 and infusion application 232 may be in communication with bus 216. Bus 216 generally provides a connection framework for these components by creating and managing all connections, providing a messaging architecture to facilitate an exchange of information between the various components of FIG. 2, and providing general operational and management capabilities for connected devices. In one embodiment, medical devices 210, 212, 214, barcode manager 224, clinical user devices 226, healthcare information system 228 and infusion application 232 communicate with bus 216 as described in U.S. patent application Ser. No. 12/347,475 (U.S. patent application '475), which is incorporated herein by reference. For example, medical devices 212, 214 might include various different types of infusion pumps that are manufactured by various different vendors. As such, components of FIG. 2 might communicate with bus 216 via a gateway (e.g., device gateway or internal gateway), an adapter, or by any other means described by U.S. patent application '475. In a further embodiment, bus 216 includes those capabilities described in U.S. patent application '475. As indicated in U.S. patent application '475, once data is received (e.g., data 218, 220, and 222) it can be sorted and routed to other applications.

In an embodiment of the present invention, such applications are included in a barcode manager 224. As such, bus 216 might receive information (e.g., data 218, 220, and 222) and route the data to barcode manager 224. Moreover, bus 216 might receive information from infusion application 232 and route the information to barcode manager 224. In a further embodiment, bus 216 receives information from healthcare information system 228 and routes the information to barcode manager 224. In another embodiment, bus 216 receives information from barcode manager 224 and routes the information to other components. For example, bus 216 routes information from barcode manager 226 to medical device 210, 212, 214.

In an embodiment of the present invention, barcode manager 224 communicates with bus 216 and functions to facilitate providing a digital barcode to a display on the medical devices 210, 212, 214 based on information received from the various components of operating environment 200 (e.g., workflows, protocols, etc.). In this way, a clinician is only provided a digital barcode on the display of the medical devices 210, 212, 214 at the appropriate point in time during the workflow. For example, information received at barcode manager 224 may indicate that although a patient's wristband has been scanned, the medication has not yet been scanned. In this case, barcode manager 224 does not allow a digital barcode to be displayed on the medical device until the medication has been scanned and communication has been established between the medical device and the EMR. This prevents the medical device from entering an error state which would require the clinician to reset the medical device and start the process of scanning the patient's wristband and the medication all over. In this way, the device only displays a digital barcode at the appropriate point in the workflow or protocol to prevent device error and allows the patient to be treated in a much more timely fashion.

Barcode manager 224 includes identification component 234, communication component 236, label component 238, and order component 240. While these components are included in the embodiment of FIG. 2, any number of components, either more or less than the illustrated components, may be used to accomplish the purposes of the present invention. Other components and subcomponents are contemplated to be within the scope of the present invention. Furthermore, although depicted as residing on one device, such as a server, it will be appreciated that any number of components and/or subcomponents may reside on any number of computing devices or servers. For example, one or more components of barcode manager 224 (e.g., label component 238) may reside on medical devices 212, 214.

Identification component 234 is generally configured to receive an identification of a patient associated with an order for an infusion. In this regard, clinical user device 226 may be a barcode scanner or a device capable of scanning a barcode (e.g., smart phone). A clinician may utilize the clinical user device 226 to scan a barcode on patient's wristband. Similarly, the clinical user device 226 and the patient's wristband (or other identifier associated with the patient) may be equipped with Bluetooth capabilities. A Bluetooth connection may be established between the clinical user device 226 and the patient's wristband so the clinical user device 226 is able to receive an identification of the patient. In another embodiment, the clinical user device 226 and the patient's wristband (or other identifier associated with the patient) may be equipped with other wireless capabilities. In this example, a wireless connection may be established between the clinical user device 226 and the patient's wristband so the clinical user device 226 is able to receive an identification of the patient. In another embodiment, the patient's wristband (or other identifier associated with the patient) may be equipped with a radio-frequency identification (RFID) tag. In this example, the clinical user device 226 may have an RFID reader so that it can read the patient's identification when the clinical user device 226 is near the patient's wristband that is equipped with the RFID tag.

Identification component 234 is also configured to receive an identification of the infusion or medication being administered to the patient by a medical device 210, 212, 214. As described above, clinical user device 226 may be a barcode scanner or a device capable of scanning a barcode (e.g., smart phone). A clinician may utilize the clinical user device 226 to scan the infusion or medication. In various embodiments, clinical user device 226 may be equipped with Bluetooth (or other wireless). In these examples, the infusion or medication (or other identifier associated with the infusion or medication) may be also be equipped with Bluetooth or other wireless capabilities. A Bluetooth or wireless connection may be established between the clinical user device 226 and the infusion or medication so the clinical user device 226 is able to receive an identification of the infusion or medication. In some embodiments, the infusion or medication (or other identifier associated with the infusion or medication) may be equipped with a radio-frequency identification (RFID) tag. In this example, an RFID reader of the clinical user device 226 may be able to read the identification of the infusion or medication when the clinical user device 226 is near the infusion or medication that is equipped with the RFID tag.

In each of the Bluetooth, wireless, and RFID examples described above, the clinician may not actually have to scan the patient's wristband or the infusion or medication. Rather, the proximity of the clinical user device 226 (by virtue of the clinician's location) to the patient (by virtue of the patient's wristband or other identifier) and/or infusion or medication identifies each of the patient and/or infusion or medication, respectively. As can also be appreciated, other location tracking and/or proximity detection mechanisms commonly used in healthcare settings may also be utilized by identification component to identify the patient and/or the infusion or medication.

Communication component 236 is generally configured to establish communication between a medical device 210, 212, 214 and an EMR 229 associated with the patient. In some embodiments, communication component 236 facilitates establishing communication between a medical device 210, 212, 214 and the EMR 229. In other embodiments, communication component 236 confirms that communication has been established between medical device 210, 212, 214 and the EMR 229. For example, communication component 226 may receive a confirmation from medical device 210, 212, 214, the EMR 229, or another component of system 200 indicating that communication has been established. Although communication is described as being between medical device 210, 212, 214 and the EMR 229, communication may be established between medical device 210, 212, 214 and another component of system 200. Communication may be made between medical device 210, 212, 214 and a component of system 200 directly, such as via bus 216, or as facilitated by some other component of system 200 not shown in FIG. 2 (such as via some other network or component in the cloud).

Label component 238 is generally configured to provide a graphical representation of a barcode (e.g., digital barcode) on a display of the medical device (e.g., infusion device). The barcode is not provided via a display (e.g., user interface) until the patient and the infusion are identified. Further, the barcode is not provided until communication between the medical device and some other component of system 200 or described herein is established. This enables the medical device to connect with the network and receive barcode details via, in one embodiment, the Bus 216, as well as enter into a state ready to receive a scan (for example, several screens of the device may not be able receive a scan, so label component 238 waits until the medical device is at a screen that can receive a scan). In this way, label component 238 confirms that the barcode is only displayed when it is needed in the workflow. This prevents any out of order scanning that may require the clinician to start the workflow over from the beginning. Once label component 238 generates the digital barcode, it can be scanned by a clinician. In some embodiments, rather than digital barcode, label component 238 may detect proximity between the clinician and the medical device (such as by Bluetooth or other wireless signal or utilizing RFID technology as described herein).

In some embodiments, label component 238 does not provide a digital barcode. For example, if one of the requirements has not yet been met (patient not identified, infusion or medication not identified, or communication not yet established, and the like), label component 238 will not provide the digital barcode. Instead, label component 238 may provide a status of the infusion workflow via a display of the medical device. In some embodiments, the status may indicate a status of the infusion workflow (e.g., waiting for patient to be scanned, waiting for infusion or medication to be scanned, establishing communication and the like). For example, the status may indicate that a connection is being established between the infusion device and an electronic medical record.

Order component 240 is generally configured to communicate the order to the medical device (e.g., infusion device). The order may be communicated from EMR 229 or another component of system 200. Order component 240 does not communicate the order to the medical device until label component 238 generates the digital barcode and it is scanned by clinician (or as proximity between the clinician and the medical device is detected). Once the order has been communicated to the infusion device, order component 240 may receive a confirmation from a clinician to begin the infusion. This may be accomplished by the clinician physically, audibly, or by gesture interacting with the medical device. In some embodiments, clinician may utilize a clinical user device 226 to confirm the order and begin the infusion.

In some embodiments, a second order is received by order component. The second order may be a modification to the first order (e.g., change in rate, dosage, and the like of an infusion). In this example, a second barcode is not presented by the user interface of the infusion device. Instead, order component 240 awaits a confirmation from the clinician, such as the confirmation described above. Once the confirmation is received, the medical device begins the infusion in accordance with the second order.

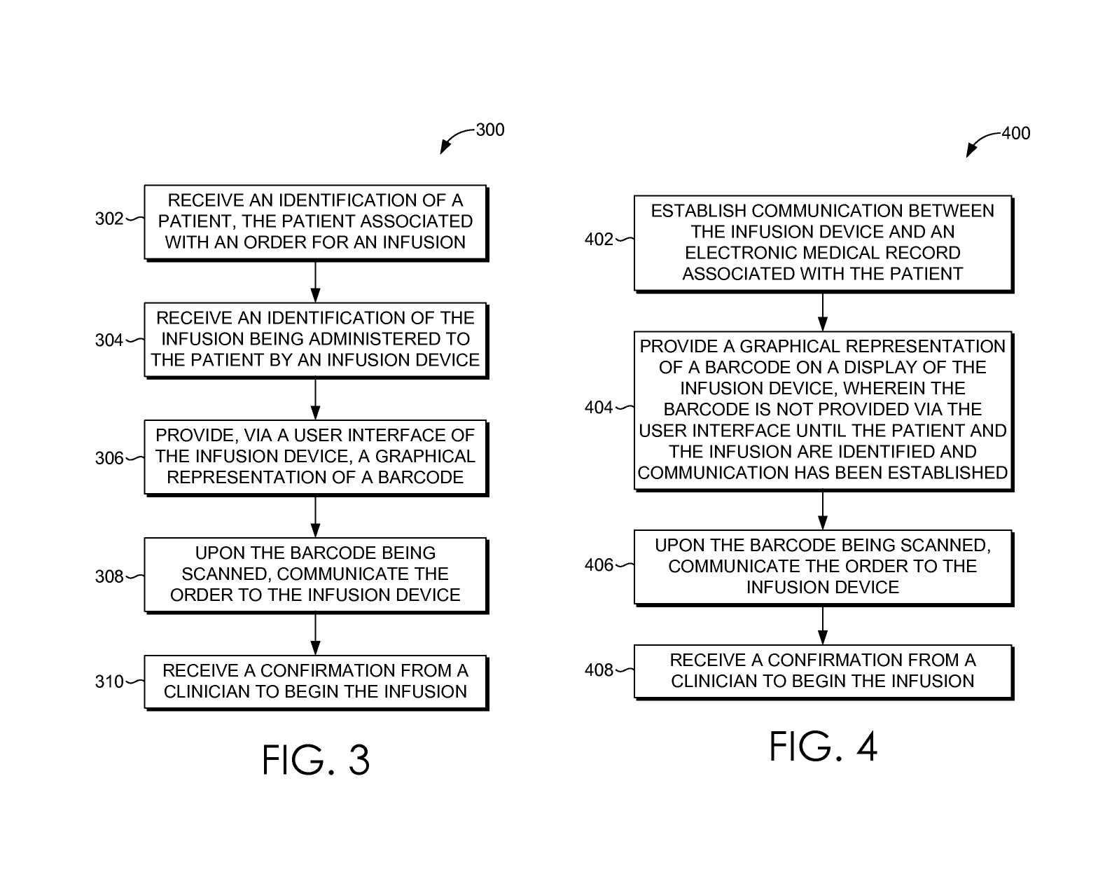

Turning now to FIG. 3, a flow diagram is provided that illustrates a method 300 for providing workflow driven digital barcodes, in accordance with embodiments of the present invention. Initially, at step 302, an identification of a patient is received. The patient may be associated with an order for an infusion. An identification of the infusion being administered to the patient by an infusion device is received at step 304. The identifications may be received via a barcode associated with the patient and a medication or infusion. In some embodiments, the identifications may be received via wireless communication or RFID tags associated with the patient and the medication or infusion. A barcode scanner, location tracking system, and/or proximity detection system may be utilized to receive the identification.

A graphical representation of a barcode (e.g., digital barcode) is provided, at step 306, via a user interface of the infusion device. The barcode is not provided via the user interface until the patient and the infusion are identified and communication has been established between the infusion device and an EMR associated with the patient. As described above, communication may be made between the infusion device and components other than the EMR. In some embodiments, the barcode is not provided via the user interface until the medical device connects with the network and receives barcode details as well as enters into a state ready to receive a scan (for example, several screens of the device may not be able receive a scan, the barcode may not be provided until the medical device is at a screen that can receive a scan).

In one embodiment, a status of the infusion workflow is provided, via the user interface, before the infusion device provides the graphical representation of the barcode (such as may be the case if the patient has not been identified, the medication or infusion has not been identified, or communication between the medical device and the EMR has not been established). The status may include that a connection is being established between the infusion device and an electronic medical record (or that the patient is being identified, or that the medication or infusion is being identified).

Upon the barcode being scanned, the order is communicated, at step 308, to the infusion device. The order may be communicated from the EMR associated with the patient. In some embodiments, rather than a barcode, proximity and/or location of the clinician, the medical device, and the medication or infusion (as well as the patient) may replace the manual scanning process. In this way, the presence of all necessary elements may cause the order to be communicated to the infusion device. At step 310, a confirmation is received from the clinician and the infusion may begin.

In some embodiments, a second order is received for a second infusion. For example, another bag may be ordered that requires a second order. In this case, a second barcode is not presented by the user interface of the infusion device.

With reference now to FIG. 4 a flow diagram is provided that illustrates a method 400 for providing workflow driven digital barcodes, in accordance with embodiments of the present invention. Initially, at step 402, communication is established between a medical device (e.g., an infusion device) and an EMR associated with a patient. Although communication is described as being a medical device and the EMR, communication may be established between medical device and another component, such as a component of FIG. 2. Communication may be made between medical device and the EMR directly (i.e., such as via bus 216 of FIG. 2) or as facilitated by some other component not shown in FIG. 2 (e.g., such as via some other network or component in the cloud).

At step 404, a graphical representation of a barcode (e.g., a digital barcode) is provided on a display of the infusion device. As described herein, the barcode is not provided via the user interface until the patient and the infusion are identified and communication has been established (between the medical device and, for example, the EMR). In some embodiments, the barcode is not provided via the user interface until the medical device connects with the network and receives barcode details as well as enters into a state ready to receive a scan (for example, several screens of the device may not be able receive a scan, the barcode may not be provided until the medical device is at a screen that can receive a scan).

In one embodiment, a status of the infusion workflow is provided before the infusion device provides the graphical representation of the barcode. For example, if the patient and the infusion have not yet been identified, or communication has not yet been established, the status may indicate the step in the workflow that is being performed (e.g., waiting for patient identification, waiting for medication or infusion identification, waiting to establish communication between the medical device and the EMR, and the like).

Upon the barcode being scanned, or the medical device being otherwise identified (e.g., via proximity, location, etc.) the order is communicated, at step 406, to the medical device. In one embodiment, the order is communicated to the medical device from the EMR. After a confirmation is received, at step 408, from the clinician, the infusion begins.

In one embodiment, a second order for a second infusion is received. The second order may be for a second bag of medication. In this case, a second barcode is not presented by the user interface of the infusion device. Instead, the second order may be confirmed by a clinician after the patient and second bag of medication has been identified.

Many different arrangements of the various components depicted, as well as components not shown, are possible without departing from the scope of the claims below. Embodiments of our technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

What is claimed is:

1. One or more computer storage media storing computer-useable instructions that, when used by one or more computing devices, cause the one or more computing devices to perform operations comprising:
receiving an identification of a patient, the patient associated with an order for an infusion;
receiving an identification of the infusion being administered to the patient by an infusion device;
providing, via a user interface of the infusion device, a graphical representation of a barcode, wherein the barcode is not provided via the user interface until the patient and the infusion are identified and communication has been established between the infusion device and an electronic medical record associated with the patient;
upon the barcode being scanned, communicating the order to the infusion device; and
receiving a confirmation from a clinician to begin the infusion.

2. The media of claim 1, further comprising establishing communication between the infusion device and the electronic medical record associated with the patient.

3. The media of claim 1, wherein the identification of the patient and the identification of the infusion are received by one of: scanning barcodes associated with the patient and the infusion or wireless communication between tags associated with the patient and the infusion and the infusion device.

4. The media of claim 1, wherein the order is communicated from an electronic medical record associated with the patient.

5. The media of claim 1, further comprising receiving a second order for a second infusion.

6. The media of claim 5, wherein a second barcode is not presented by the user interface of the infusion device.

7. The media of claim 1, further comprising providing, via the user interface, a status of the infusion workflow before the infusion device provides the graphical representation of the barcode.

8. The media of claim 7, wherein the status includes establishing a connection between the infusion device and an electronic medical record.

9. A computer-implemented method in a clinical computing environment comprising:
establishing, via a first computing process, communication between an infusion device and an electronic medical record associated with a patient;
providing, via a second computing process, a graphical representation of a barcode on a display of the infusion device, wherein the barcode is not provided via the user interface until the patient and the infusion are identified and communication has been established;
upon the barcode being scanned, communicating, via a third computing process, the order to the infusion device; and
receiving, from a fourth computing process, a confirmation from a clinician to begin the infusion;
wherein each computing process is performed by one or more computing devices.

10. The media of claim 9, wherein the order is communicated from the electronic medical record associated with the patient.

11. The media of claim 9, further comprising receiving, via a fifth computing process, a second order for a second infusion.

12. The media of claim 11, wherein a second barcode is not presented by the user interface of the infusion device.

13. The media of claim 9, further comprising providing, via a sixth computing process, a status of the infusion workflow before the infusion device provides the graphical representation of the barcode.

14. The media of claim 13, wherein the status includes establishing a connection between the infusion device and an electronic medical record.

15. A system comprising:
one or more processors; and
one or more computer storage media storing instructions that, when used by the one or more processors, cause the one or more processors to:
receive an identification of a patient, the patient associated with an order for an infusion;
receive an identification of the infusion being administered to the patient by an infusion device;
establish communication with the infusion device and an electronic medical record associated with the patient;
provide a graphical representation of a barcode on a display of the infusion device, wherein the barcode is not provided via a user interface until the patient and the infusion are identified and communication has been established;
upon the barcode being scanned, communicate the order to the infusion device; and
receive a confirmation from a clinician to begin the infusion.

16. The system of claim 15, wherein the order is communicated from the electronic medical record associated with the patient.

17. The system of claim 15, further comprising the one or more computer storage media storing instructions that, when used by the one or more processors, cause the one or more processors to receive a second order for a second infusion.

18. The system of claim 17, wherein a second barcode is not presented by the user interface of the infusion device.

19. The system of claim 15, further comprising the one or more computer storage media storing instructions that, when used by the one or more processors, cause the one or more processors to provide a status of the infusion workflow before the infusion device provides the graphical representation of the barcode.

20. The system of claim 19, wherein the status includes establishing a connection between the infusion device and an electronic medical record.
